(19) United States
(12) Patent Application Publication
(10) Pub. No.: US 2023/0272928 A1
(43) Pub. Date: Aug. 31, 2023

(54) Pollution Control Devices Methods and Systems

(71) Applicant: Oy Halton Group Ltd., Helsinki (FI)

(72) Inventors: Andrey V. LIVCHAK, Bowling Green, KY (US); Gregory A. LYONS, Tompkinsville, KY (US); Ben ARNOLD, Bowling Green, KY (US); Jimmy SANDUSKY, Bowling Green, KY (US); Philip J. MEREDITH, Alvaton, KY (US)

(73) Assignee: Oy Halton Group Ltd., Helsinki (FI)

(21) Appl. No.: 18/009,086

(22) PCT Filed: Jun. 28, 2021
(86) PCT No.: PCT/US21/39353

§ 371 Date: Dec. 8, 2022

Related U.S. Application Data
(60) Provisional application No. 63/049,569, filed on Jul. 8, 2020.

(51) Int. Cl. F24F 8/22; A61L 9/18; F24F 13/06; F24F 7/06; F24F 11/89

(52) U.S. Cl. CPC F24F 8/22; A61L 9/18; F24F 13/06; F24F 7/065; F24F 11/89; A61L 2209/111; A61L 2209/14

(57) ABSTRACT

A ventilation system return register is mountable in a room from with air will flow through the return register. It includes a housing that defines an internal volume through which air can flow, a transition collar configured to attach to the ventilation system duct, an electrical junction box configured to receive an electrical connection and a disinfecting light source electrically connected to the electrical junction box and configured to emit a radiation in a range that destroys microbial contaminants A reflective surface faces the disinfecting light and defines a disinfecting pathway through which air passes when the ventilation system is operating while it prevents the disinfecting light from shining into the room.

### CROSS-REFERENCE TO RELATED APPLICATIONS

This application claims priority to and the benefit of U.S. Provisional Patent Application No. 63/049,569 filed Jul. 8, 2020, which is hereby incorporated by reference in its entirety.

### BACKGROUND

It is desirable to disinfect or sanitize air and spaces occupied by humans to reduce the quantity or eliminate entirely various pathogens, such as viruses and bacteria, that may be present in the space and the air around the space. This disclosure relates to devices and systems that generally address these needs, and others, sometimes employing ultraviolet (UV) radiation to filter or decontaminate occupied spaces and/or air in those spaces. The term sanitize will be understood to be interchangeable with disinfect in this disclosure. The terms kill, inactivate, and destroy will be used interchangeably herein to refer to the effect of UV radiation on bacteria, viruses, and other pathogens, and are intended to all generally describe the reduction of potency, the reduction of infectiveness, the reduction in the effectiveness, the reduction of the quantity, and/or the elimination entirely of the pathogens after they have been exposed to the radiation.

### SUMMARY

One or more embodiments of the disclose subject matter sanitize and disinfect air in a room and also provide the ability to sanitize and disinfect surfaces in the room where the embodiments are installed.

Objects and advantages of embodiments of the disclosed subject matter will become apparent from the following description when considered in conjunction with the accompanying drawings.

### DESCRIPTION OF THE DRAWINGS

Embodiments will hereinafter be described in detail below with reference to the accompanying drawings, wherein like reference numerals represent like elements. The accompanying drawings have not necessarily been drawn to scale. Some of the figures may have been simplified by the omission of selected features for the purpose of more clearly showing other underlying features. Such omissions of elements in some figures are not necessarily indicative of the presence or absence of particular elements in any of the exemplary embodiments, except as may be explicitly disclosed in the corresponding written description.

FIG. 1A illustrates a schematic representation of a dual-mode light fixture in an occupied mode according to embodiments of the disclosed subject matter.

FIG. 1B illustrates a cross-sectional view of the dual-mode light fixture of FIG. 1A taken along line B-B.

FIG. 2A illustrates a schematic representation of a dual-mode light fixture in an unoccupied mode according to embodiments of the disclosed subject matter.

FIG. 2B illustrates a cross-sectional view of the dual-mode light fixture of FIG. 2A taken along line B-B.

FIGS. 2C and 2D illustrate a schematic representation of a dual-mode fixture according to embodiments of the disclosed subject matter.

FIG. 3A illustrates a cross-sectional view of a dual mode light fixture according to embodiments of the disclosed subject matter.

FIG. 3B illustrates a cross-sectional view of a dual mode light fixture according to further embodiments of the disclosed subject matter.

FIG. 3C illustrates a cross-sectional view of a component of a light fixture according to further embodiments of the disclosed subject matter.

FIG. 3D illustrates a schematic representation of a light socket used with various embodiments of the disclosed subject matter.

FIG. 4A illustrates a schematic representation of a surface-mounted sanitizing unit in an occupied mode according to embodiments of the disclosed subject matter.

FIG. 4B illustrates a schematic representation of the surface-mounted sanitizing unit of FIG. 4A in an unoccupied mode.

FIG. 5 illustrates a schematic representation of a disinfecting air return grille according to embodiments of the disclosed subject matter.

FIG. 6A illustrates a schematic representation of a disinfecting air return grille according to further embodiments of the disclosed subject matter.

FIG. 6B illustrates a blown-up schematic representation of the disinfecting air return grille according of FIG. 6A.

FIG. 7 illustrates an example of control logic for various embodiments of the disclosed subject matter.

FIG. 8 illustrates a schematic representation of a disinfecting system according to embodiments of the disclosed subject matter.

FIG. 9 illustrates a method of controlling a dual-mode fixture according to embodiments of the disclosed subject matter.

FIG. 10 illustrates an exemplary implementation of the controllers used in various embodiments of the disclosed subject matter.

### DETAILED DESCRIPTION

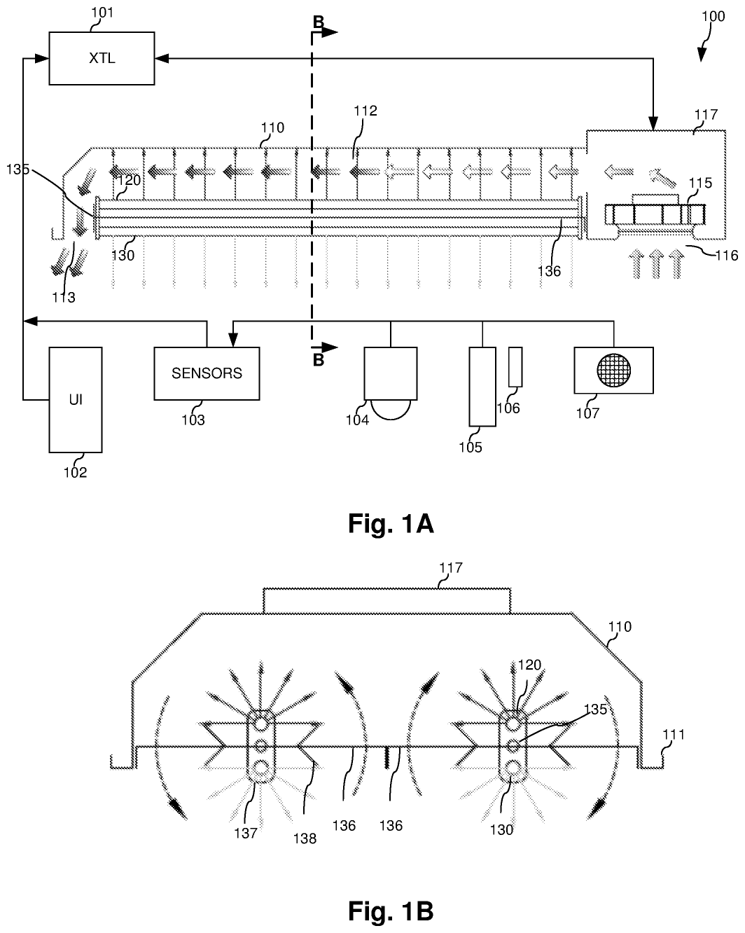

Referring to FIG. 1A, a dual-mode light fixture 100 is illustrated in a schematic representation. This particular illustration represents what will be referred to as the occupied mode, where the space below the dual-mode light fixture 100 is expected to be occupied by people.

The dual-mode light fixture 100 may be mounted on a ceiling or inside a suspended ceiling, but may also be mounted in other positions and locations. In an embodiment, dual-mode light fixture 100 is installed in an enclosed space, on the ceiling. The dual-mode light fixture 100 includes a housing 110, as shown in the drawings. In some embodiments, the housing 110 is elongate such that its length is greater than its height and width. Although FIG. 1A is not drawn to scale, the general elongate shape of the housing 110 can be appreciated.

The dual-mode light fixture 100 further includes an intake plenum 117 and a disinfecting plenum 112, which are both housed within the housing 110. The intake plenum 117 is a space in which a fan 115 is housed. The fan 115 is driven by a motor (not shown) and draws air through air inlet 116 into the intake plenum 117. The air then continues from the intake plenum 117 into disinfecting plenum 112. When the dual-mode light fixture 100 is installed in a ceiling 401 and viewed from the ground, the air inlet 116 would be visible at one end of the dual-mode light fixture 100, while air outlet 113 would be visible at the opposite end of dual-mode light fixture 100.

The disinfecting plenum 112 is bounded by a portion of the housing 110 from above, and by a base plate 136 from the bottom. The base plate 136 is better seen in FIG. 1B, which shows a cross-sectional view along line B-B in FIG. 1A. There may be more than one base plate 136, such as two plates 136, which together form the floor of the disinfecting plenum 112. The base plate 136 is rotatably mounted so that it can pivot about pivot 135, as is indicated by the dashed arrows in FIG. 1B. On the surface of the base plate 136 are mounted two types of lights. On one surface (the top surface in FIG. 1A) is mounted a disinfecting light source, such as UV light 120. In embodiments, the UV light 120 emits light in the frequency range of 240-280 nm, which has been found to destroy various germs and viruses.

On the opposite surface (the bottom surface in FIG. 1B) of the base plate 136 is mounted a visible light source which emits light in the visible spectrum, such as visible light 130. In embodiments, UV light 120 and visible light 130 have a shape profile of fluorescent tubes that can be mounted parallel to the rotation axis of pivot 135. The UV light 120 and visible light 130 can be held by light socket 137. The light socket 137 may have a common housing that protrudes through base plate 136 such that it can hold both types of lights and provide power to the lights. In other embodiments, a separate light socket 137 is provided on each side of the base plate 136.

Lights 130 are not limited to any particular light format, and could be fluorescent light tubes, incandescent lights, light emitting diodes (LEDs), or any other components that emit visible light. In some cases, lights 130 may be fluorescent lights and may share a ballast (not illustrated) with the UV lights 120. It will be understood that lights 120 may have separate ballast(s) from the ballast(s) of UV lights 120.

Still referring to FIG. 1A, the dual-mode light fixture 100 can be connected to a controller 101, which is additionally or alternatively connected to sensors 103 and user interface 102. The user interface 102 may include switches or toggles that are operable by people in the occupied space in which the dual-mode light fixture 100 is installed, and may further include a display and/or warning lights to indicate the operation of the dual-mode light fixture 100 in certain modes.

The sensors 103 generally provide the ability to detect the presence and confirm the absence of people in the occupied space where the dual-mode light fixture 100 is installed. For example, IR sensor 104 may be passive or active infrared sensor that detects changes in an infrared image and thereby identifies motion. The door sensor 105 may include a reed switch and can be mounted near a door and detect opening and closing of the door when magnet 106 is mounted on the door. Acoustic sensor 107 may detect sounds and apply a classifier to the sounds to identify presence of people in the occupied space. Other sensors, though not illustrated, may be employed in addition or instead of those that are shown, to detect the presence and absence of people in the occupied space.

Referring now to FIG. 1B, a cross sectional view of the dual-mode light fixture 100 can be seen. It can be more readily appreciated that the base plate 136 can rotate to expose the UV light 120 to the disinfecting plenum 112 while at the same time exposing visible light 130 to the occupied space during the occupied mode. Further, mounting flange 111 is also shown in FIG. 1B and generally illustrates how the dual-mode light fixture 100 may be mounted in a surface, such as a ceiling or a wall.

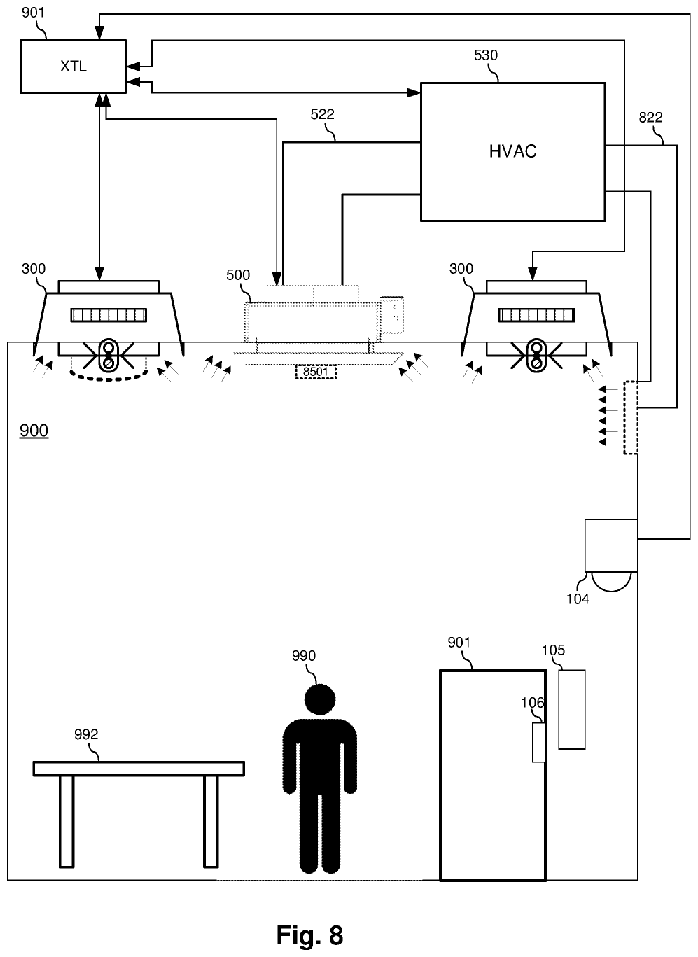

The occupied mode is understood as a mode of operation of dual-mode light fixture 100 when people are present or expected to be present in the occupied space where the dual-mode light fixture 100 is installed. In exemplary embodiments, the occupied space may be an enclosed space such as a room or an office (such as shown in FIG. 8.) In this mode, the visible light 130 outputs visible light that shines from the dual-mode light fixture 100 into the occupied space. The visible light may be emitted at one or more color temperatures based on the type of specific light used as visible light 130. For example, visible light 130 may be a fluorescent tube or an LED tube.

At the same time, the fan 115 operates (as controlled by the controller 101) to draw air from the occupied space through air inlet 116, past intake plenum 117, into disinfecting plenum 112. In the disinfecting plenum 112, the UV light 120 is activated and radiates UV radiation which is then reflected from the upper wall of the disinfecting plenum 112 (which is a part of the housing 110) and reflected again from the base plate 136. The base plate 136 may further include reflector(s) 138 that help to direct the UV radiation from the UV light 120. It will be appreciated that the disinfecting plenum 112 is subject to UV radiation and the intensity of the UV radiation can be controlled in various ways (e.g., selecting the type of UV light 120; selecting the quantity of UV lights 120; selecting the length of the disinfecting plenum 112, etc.) and the airflow speed through the disinfecting plenum 112 can be controlled.

As the air flows through the disinfecting plenum 112, any airborne pathogens, pollutants, germs, and viruses are subjected to the UV radiation. Once the air exits the disinfecting plenum 112 through air outlet 113, it is considered to be sanitized air, which is then returned to the occupied space. It will be understood that this sanitized air mixes with other air in the occupied space, and can be repeatedly pulled into air inlet 116 to be repeatedly sanitized. In this manner, the quality of air in the occupied space where the dual-mode light fixture 100 is installed can be continuously improved. It should be further appreciated that multiple dual-mode light fixture 100 can be installed in one room and thus can work in concert with each other to sanitize the air in the room. For example, all standard light fixtures may be replaced with the dual-mode light fixture 100.

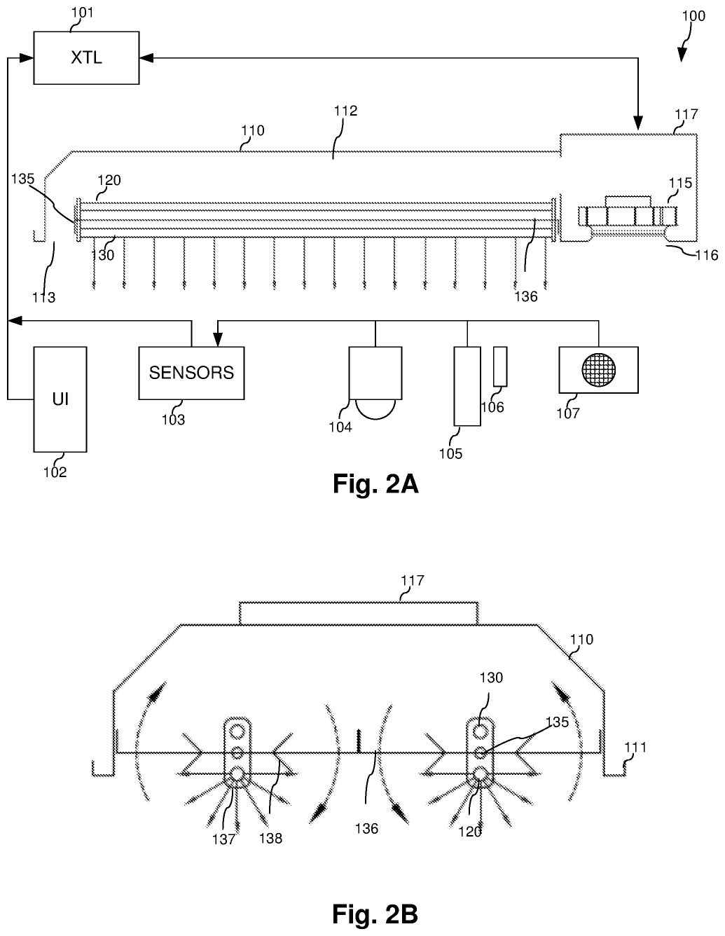

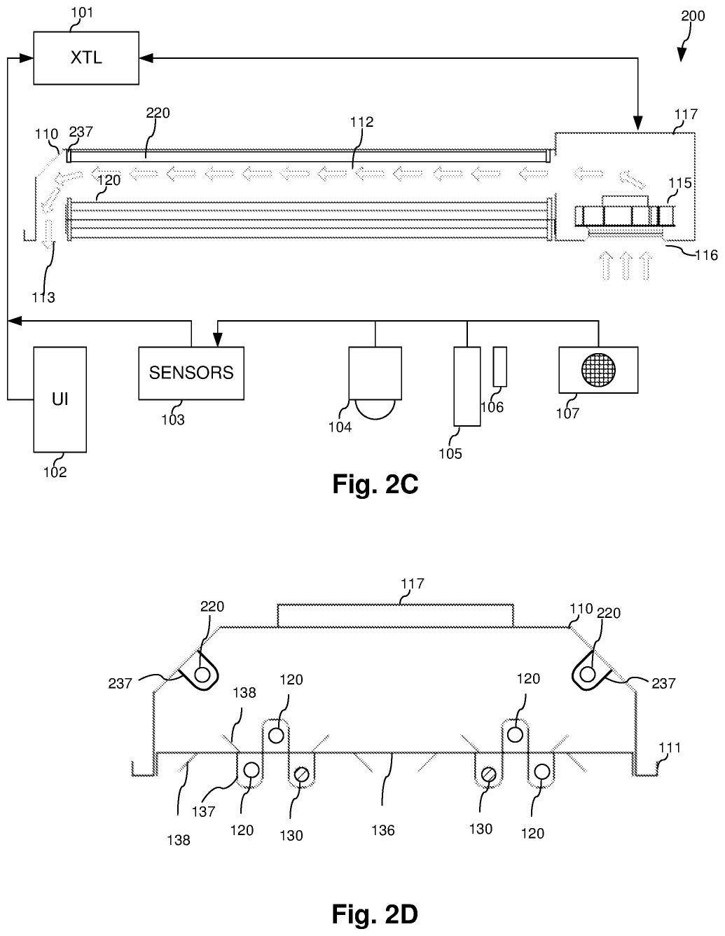

Turning next to FIGS. 2A-B, the dual-mode light fixture 100 is now shown in the unoccupied mode. In this mode, the base plate 136 has rotated such that the UV light 120 is now exposed to the room below, while the visible light 130 is now inside the disinfecting plenum 112. It will be appreciated that the visible light 130 will generally be turned off in this mode, while the UV light 120 is turned on and radiates UV radiation into the occupied space where the dual-mode light fixture 100 is installed. This can expose surfaces near the dual-mode light fixture 100 to UV radiation, which can help sanitize those surfaces. The base plate 136 may have one or more reflectors 138 to help direct the UV radiation in desired directions.

It may be undesirable to expose human occupants to the UV radiation during the unoccupied mode. The controller 101 can receive signals from various sensors 103 to monitor continuously or periodically whether the space near the dual-mode light fixture 100 is free of people. In embodiments, the controller 101 may use a timer to automatically switch between the occupied mode and the unoccupied mode. In some embodiments, the unoccupied mode may be entered at a specified time of day and/or after a predetermined amount of time has passed since the sensors 103 have detected the presence of human occupants in the space near dual-mode light fixture 100.

As a safety measure, the controller 101 can turn off the UV light 120 when the dual-mode light fixture 100 is in the unoccupied mode when the controller 101 determines that human occupants have entered the space. In embodiments, when the door sensor 105 indicates opening of a door, the UV light 120 is turned off to prevent the human opening the door from exposure to UV light. It will be appreciated that that other sensors may be employed in a similar fashion to avoid exposing humans entering the space to UV light.

The user interface 102 may output a visible and/or audible warning during the unoccupied mode that UV radiation is being used to sanitize surfaces. Multiple output modules may be placed inside and outside of the room where the dual-mode light fixture 100 is installed to provide warning. In embodiments, a visible warning can be generated near door(s) that lead to the space to inform human occupants of the sanitizing operation and to discourage entry.

Referring to FIGS. 2C-D, an alternate embodiment of dual mode light fixture 200 is shown. In this exemplary embodiment, the two modes—occupied mode and unoccupied mode—do not require any movement of the base plate 136. Instead, the two modes are selected my controlling which lights are turned on. As can be seen in FIG. 2D, one or more visible lights 130 may be installed facing to the exterior of the light fixture. In this example, two lights 130 are shown, but there could be more or fewer such lights. One or more UV lights 120 are installed facing to the exterior (down in FIG. 2D) and one or more UV lights 120 are installed facing into the disinfecting plenum 112. Additional UV lights 220 may be mounted via light sockets 237 at locations other than the base plate 136, as shown in FIG. 2D. The UV lights 220 are shown as mounted on the beveled portion of housing 110, but the location is not so limited. Further, UV lights 220 may be present in all of the dual light fixture embodiments described herein, although they may be omitted in certain drawings. The presence of additional UV lights 220 can increase the effectiveness of the disinfecting operation inside the disinfecting plenum 112. It will also be understood that UV lights 120 could be omitted when UV lights 220 are installed, while still providing the dual mode functionality.

When the light fixture 200 is in the occupied mode, any UV lights (120 and/or 220) that are on the interior of the disinfecting plenum 112 are turned on and the fan 115 operates to draw room air through the disinfecting plenum 112 and to discharge the disinfected air through air outlet 113. In this mode, the visible light 130 is powered on, thus providing visible light below the dual mode light fixture 200, while any UV lights 120 that are face the occupied space are turned off.

When the light fixture 200 is in the unoccupied mode, the visible lights 130 are turned off, and all of the UV lights 120 and 220, whether installed in the disinfecting plenum or facing to the exterior are turned on, and the fan 115 is also turned on. The speed of the fan 115 may be increased over the speed that is used in the unoccupied mode, in some embodiments. This may result in a larger volume of air being circulated through the disinfecting plenum, with a possible increase in fan noise due to the increased air flow rate. But the increased noise will be acceptable as the room where the light is installed in expected to be unoccupied. The UV lights 120 that face to the exterior of the light fixture 200 will illuminate the surfaces below the light fixture 200 with UV light, disinfecting the surfaces.

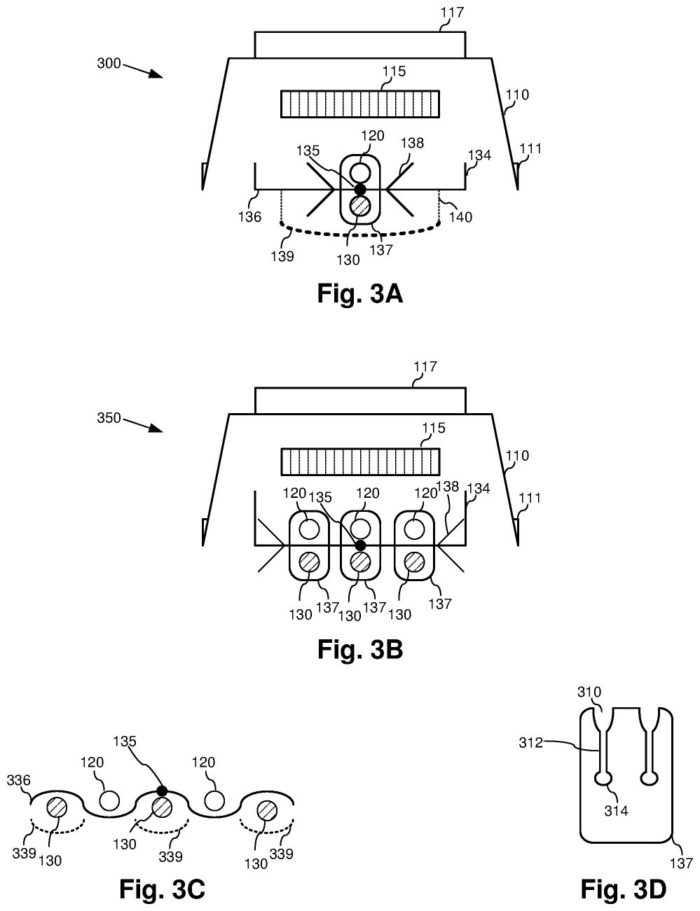

Turning now to FIG. 3A, an alternate embodiment of dual-mode light fixture 300 is shown. While dual-mode light fixture 300 contains many of the same elements as dual-mode light fixture 100, it only contains a single base plate 136, which has guard walls 134 on sides of the base plate 136. The guard wall 134 are directed away from the base plate 136 and create a barrier that blocks UV light from shining out of dual-mode light fixture 300 during the occupied mode (where the UV light 120 is turned on and irradiating air in the disinfecting plenum 112). The guard wall 134 will allow a bluish glow to be seen from the space near the dual-mode light fixture 300, but will block harmful quantity of the UV radiation from reaching any human occupants in the space.

As shown in FIG. 3A, any of the embodiments of the dual-mode light fixture may include one or more diffuser 139. In the illustrated embodiment, the diffuser 139 is positioned in front of the visible light 130 to diffuse the visible light being emitted, thus reducing harsh shadows and hot spots. The diffuser 139 may be a mesh with a regular pattern of holes or other similar materials, and can be mounted to the base plate 136 with diffuser supports 140.

Turning to FIG. 3B, another embodiment of dual-mode light fixture 350 is shown. In this embodiment, a single base plate 136 holds three pairs of lights—three UV lights 120 on one side and three visible lights 130 on the other side. It should be apparent that any of the embodiments of the base plate 136 can be combined together. For example, the dual-mode light fixture 100 of FIGS. 1A-2B could use a single base plate 136 with one or more of each of UV light 120 and visible light 130, or more than one, such as two, three, or more than three of base plate 136, each base plate 136 having a different number of lights on it.

Although base plate 136 has thus far been described and illustrated as flat plate, it is not so limited. Referring to FIG. 3C, the base plate 336 has a scalloped shape with alternating troughs and peaks (shown with a rounded profile, but not limited to such a shape). The alternating troughs and peaks could have a more triangular profile which may produce a different light pattern. The embodiment in FIG. 3C also includes diffusers 339, but they may be omitted. It will be apparent that the base plate 336 may be used in any of the embodiments of the dual-mode light fixture 100, 300, and 350 discussed above.

FIG. 3D illustrates additional details of an exemplary embodiments of light socket 137 which would be present in various embodiments of the base plate 136. The light socket 137 is designed to hold a light, such as a fluorescent tube. To that end, it has two slots 312 which being with mouth 310 and end with a recess 314. A light tube may have electrical terminals that extend from the end of the light tube and the terminals fit into the slots 312, and become electrically connected to a power source when the terminals reach recess 314.

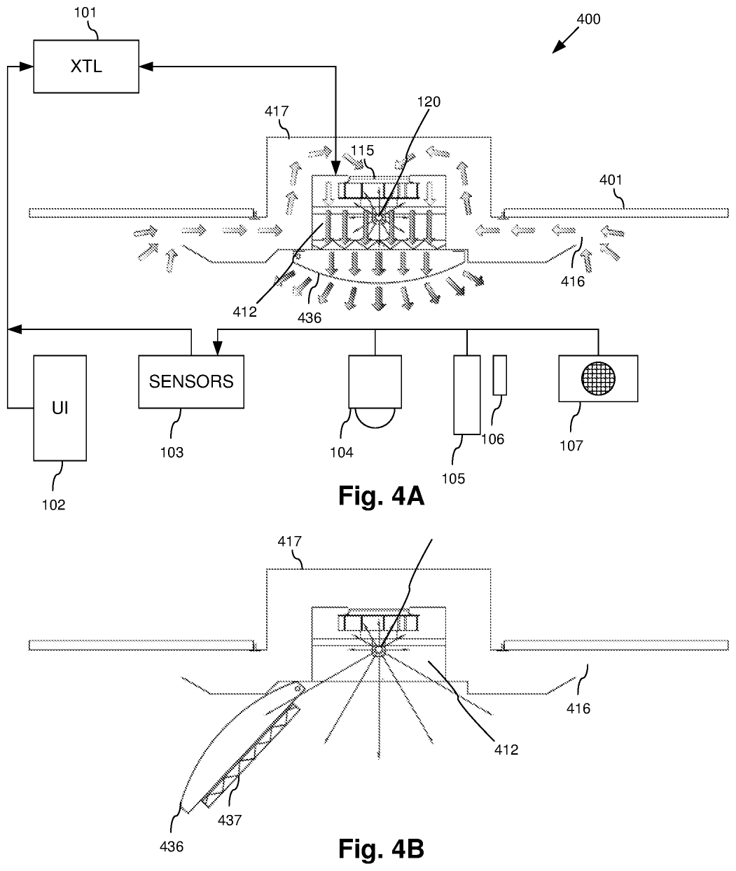

Referring to FIG. 4A, an embodiment of an air sanitizer 400 is shown as it may be installed in a ceiling 401 of a room. Similar in some respects to dual-mode light fixture 100, the air sanitizer 400 has an air inlet 416 through which air is drawn by fan 115 into intake plenum 417. From the intake plenum 417 the air passes into disinfecting plenum 412 which is downstream of the fan 115 and is irradiated by UV light 120. The air sanitizer 400 may also have a filter 437 in the path of the air flow, such that the air is not only irradiated by UV light, but also filtered by the filter 437. The filter 437 may be a HEPA filter of a various ratings selected for a particular situation. It will be appreciated that the filter 437 may physically trap pathogens that are present in the air and over time may become filled with such pathogens. The presence of the UV light 120 helps destroy the pathogens, such as bacteria and viruses, that might be trapped in the filter 437, such that the possibility of further spreading the pathogens from the filter 437 is reduced or eliminated. In this manner, the air sanitizer 400 disinfects and sanitizes air.

It will be understood that in FIG. 4A the air sanitizer 400 is operating in the occupied mode, such that space in the vicinity of the air sanitizer 400 can be safely occupied by humans, because the UV light from UV light 120 is not emitted outside of the air sanitizer 400.

FIG. 4B illustrates the air sanitizer 400 in the unoccupied mode, in which surfaces in the vicinity of the air sanitizer 400 are sanitized and disinfected. In the unoccupied mode, the cover grille 436 of the air sanitizer 400 swivels to an opened position, exposing the UV light 120 to the environment and allowing UV radiation from the UV light 120 to shine onto surfaces facing the air sanitizer 400. As described above, the UV radiation can destroy and inactivate various pathogens including viruses and bacteria. The cover grille 436 may be mechanically linked to an actuator (not shown) that moves the cover grille 436 between the two positions.

Similar to other embodiments, the air sanitizer 400 is controlled by controller 101 to turn off the UV light 120 in the event that a human enters the space in the vicinity of the air sanitizer 400 by relying on signals from sensors 103.

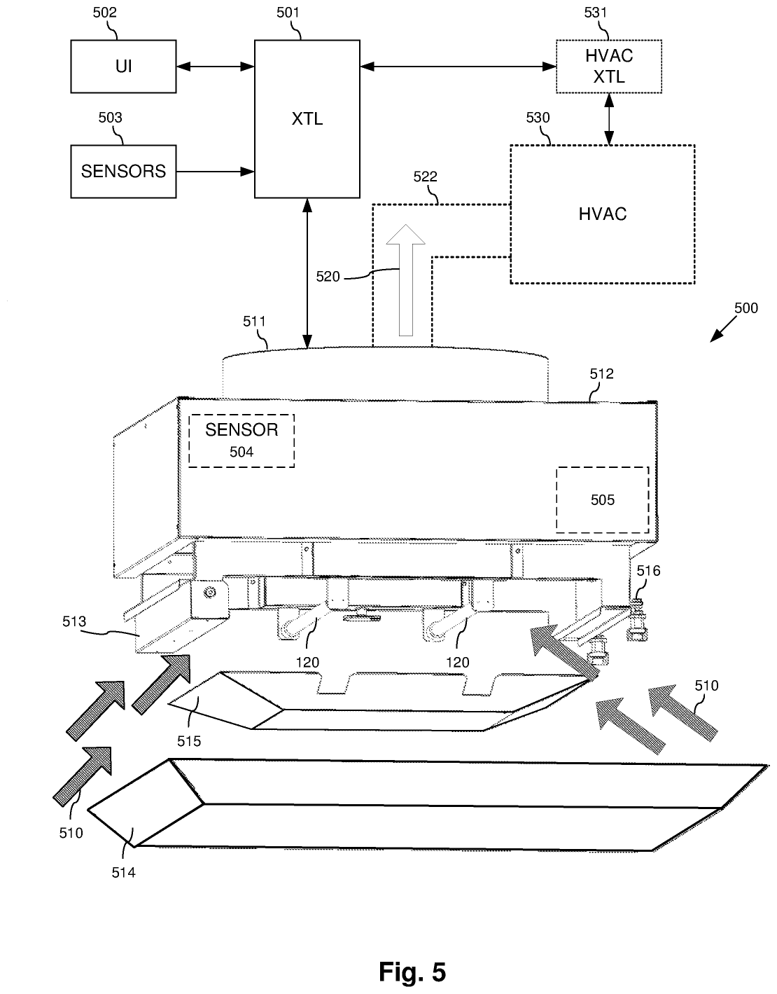

Referring to FIG. 5, a disinfecting return grille 500 according to embodiments of the disclosure is shown. The disinfecting return grille 500 is installed at the end of a ventilation duct 522 (which serves as a return duct) for HVAC system 530. The HVAC system 530 generally cycles air from an indoor space through an air conditioning operation (that may include heating and/or cooling of air) and then returns the conditioned air back into the indoor space via supply duct 822 (see, e.g., FIG. 8). The air is thus recycled, so the same gas molecules can be thought of repeatedly going into the indoor space and then again out of the indoor space. It is apparent that there is a benefit in cleaning the air to improve air quality.

HVAC systems often have sanitizing and disinfecting functionality that cleans air as part of the air conditioning (which includes heating and/or cooling) operation, to output clean air. HVAC system 530 may have air cleaning and sanitizing functionality using one or more filters, germicidal light(s), chemical disinfectants, and corona discharge wires. However, any cleaning taking place in HVAC system 530 does not affect air that has just entered the ventilation duct 522 as it is pulled from the indoor space. One source of possible pathogens are humans occupying the space and exhaling air (room air 510) that may contain viruses and bacteria. It is beneficial to inactivate those pathogens as close to the source (i.e., the human occupants) before they can settle in the ventilation ductwork.

The disinfecting return grille 500 is designed to sanitize and disinfect room air 510 before it enters ductwork that conveys the return air to a central HVAC system 530, so that sanitized air 520 is provided to the ventilation duct 522, thus addressing the above need. The disinfecting return grille 500 has a plenum box 512 that serves as a housing that may be fully or partially placed in a mounting surface (such as a ceiling or a wall). At one end of the plenum box 512 is a transition collar 511 which is sized and shaped to be connected to the return ductwork, such as ventilation duct 522 of the HVAC system 530. The transition collar 511 is illustrated as having a circular shape, but it is not so limited and any shape is possible so that it can mate with the ventilation duct 522.

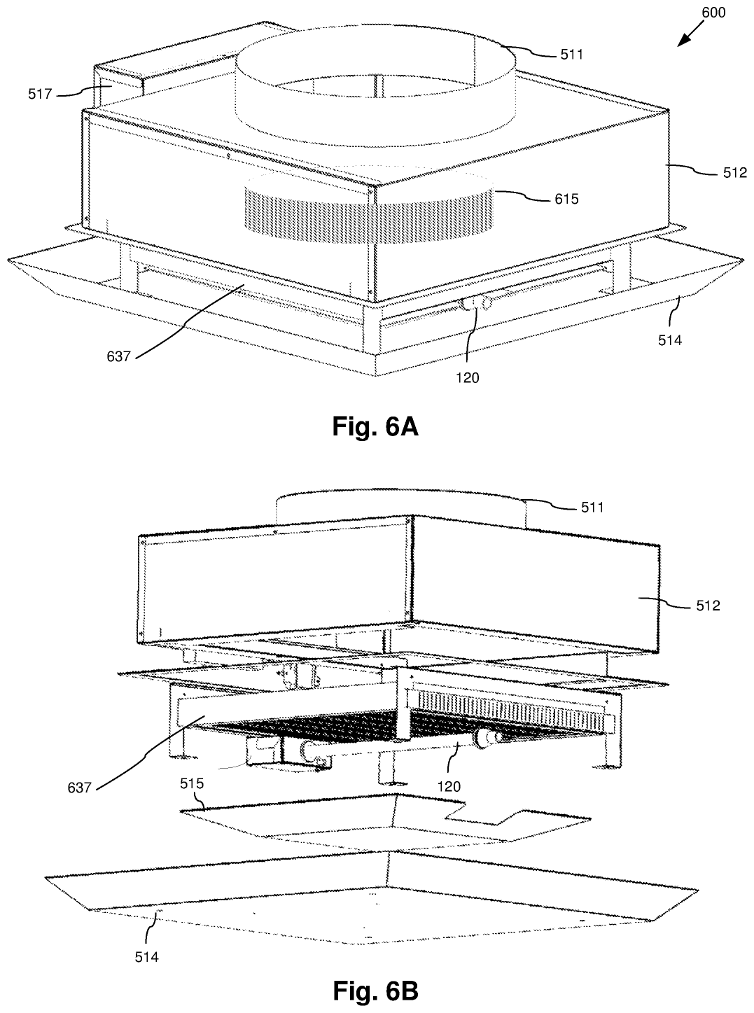

Inside of plenum box 512 may be one or more sensors such as a pressure sensor 504 and optionally a powered fan 615 (see FIG. 6A). At an end opposite the transition collar 511 is mounting hardware that holds one or more UV lights 120 and a reflector 515. The UV light 120 is connected to an electrical connection (not shown) and can be turned on and off by control signals. FIG. 5 shows a partially exploded views, but it will be understood that reflector 515 is positioned closely adjacent to plenum box 512 to create a sanitizing air flow path through which room air 510 is pulled into the disinfecting return grille 500. The disinfecting return grille 500 may also include an intake plate 514 which may have holes, slots, or other openings that permit room air 510 to enter the disinfecting return grille 500.

The plenum box 512 may include a pivot bracket 513 to which the intake plate 514 can be mounted, thereby providing the ability for the intake plate 514 to pivot away from the plenum box 512. In this embodiment, the reflector 515 may also be mounted to the intake plate 514, so that when the intake plate 514 pivots away from the plenum box 512, the UV lights 120 are exposed to the environment.

In embodiments, the plenum box 512 may also include a plate switch 505, which can sense whether the intake plate 514 is opened or closed. This plate switch 505 can serve as a safety measure to prevent turning on UV lights 120 when the intake plate 514 is opened, to avoid irradiating people in the room below the disinfecting return grille 500. As explained later, in other embodiments the disinfecting return grille 500 may be used for a surface sanitizing operation (much like the dual-purpose light fixtures described above), in which case the plate switch 505 will not disable the operation of the UV lights 120.

It will be understood that when the UV lights 120 are exposed to the environment, a surface sanitizing operation can take place. In this manner, the disinfecting return grille 500 can operate in an occupied mode and an unoccupied mode, akin to the dual-mode light fixtures described above.

The system that includes the disinfecting return grille 500 and HVAC system 530 includes a controller 501 that controls the disinfecting return grille 500 and may also control the HVAC system 530 or communicates with a dedicated HVAC controller 531. The system may also include a user interface 502 and sensors 503, similar to the user interface and sensors already described above.

An example of the operation of the disinfecting return grille 500 will now be described. During occupied mode, which may be based on a time schedule, such as business hours, the disinfecting return grille 500 receives room air 510, which passes through UV radiation emitted by UV light 120, and passes through plenum box 512 and transition collar 511 to the ventilation duct 522 as sanitized air 520. The sanitized air 520 travels to the HVAC system 530 where it is conditioned and returned to the indoor space.

The occupied mode can also be based on sensor output, such as output from sensors 503 that indicate that the room is occupied. In embodiments, the occupied mode can be selected based on output from a pressure sensor inside plenum box 512, that detects air flow caused by the HVAC system 530. When HVAC system 530 is operating, it draws air in through ventilation duct 522, which generates a detectable pressure change in plenum box 512. Thus, when the HVAC system 530 is drawing air through ventilation duct 522, the disinfecting return grille 500 can operate in the occupied mode, sanitizing room air 510 that is drawn into the disinfecting return grille 500.

The occupied mode can end based on a timer, an input from the user interface 502, and/or based on a detection that the HVAC system 530 has turned off. In some buildings, the HVAC system 530 turns off during certain hours, which are expected to be times when the building is not occupied. Then, the unoccupied mode may begin.

In the unoccupied mode, the intake plate 514 and reflector 515 can flip down to expose the UV light 120 to the environment. The intake plate 514 may be held by attachment bolts 516 as shown in FIG. 5, but may also be moved by a different mechanism, such as a stepper motor or another actuator (not shown). In the unoccupied mode, the UV light 120 emits UV light toward surfaces in the indoor space, inactivating pathogens that are sensitive to UV light.

The controller 501 monitors output from sensors 503 to ensure that the indoor space remain unoccupied. If the sensors 503 output signals that indicate the presence of a human occupant, the controller 501 turns off the UV light 120 to avoid exposing the occupant to UV light.

Turning to FIGS. 6A and 6B, a disinfecting grille 600 is shown with an optional filter 637. While FIG. 6A does not illustrate a pivoting mechanism for the intake plate 514, it is understood that the disinfecting grille 600 can also operate in two modes—an occupied mode and an unoccupied mode. FIG. 6B illustrates a partially exploded view of the disinfecting grille 600 to illustrate the spatial relationships more accurately between the various components.

The filter 637 can further clean room air 510 before it enters the ventilation duct 522 by removing pollutants that might not be inactivated by UV light. However, the filter 637 may over time trap bacteria and other pollutants that could, without intervention, grow on the filter 637. A UV light 120 (or multiple such lights) is provided immediately adjacent to filter 637 and serves the dual purpose of sanitizing air passing near the UV light 120, and also continuously irradiating the filter 637 to reduce or stop growth of bacteria and mold (and other pathogens) on the filter 637.

The disinfecting grille 600 may include a powered fan 615 as shown. The fan 615 and the UV light 120 can be connected to a source of electricity through electrical junction housing 517. When the disinfecting grille 600 is installed as a retrofit for a standard air return grille, the sanitizing functionality of the disinfecting grille 600 may cause a pressure drop due to the filter and/or a tortuous path for room air 510 as it traverses the sanitizing features. The fan 615 can be configured to counteract this pressure drop and to provide an air flow from the intake plate 514 to the transition collar 511 at a flow rate that compensates for the addition of the filter 637 and the sanitizing features.

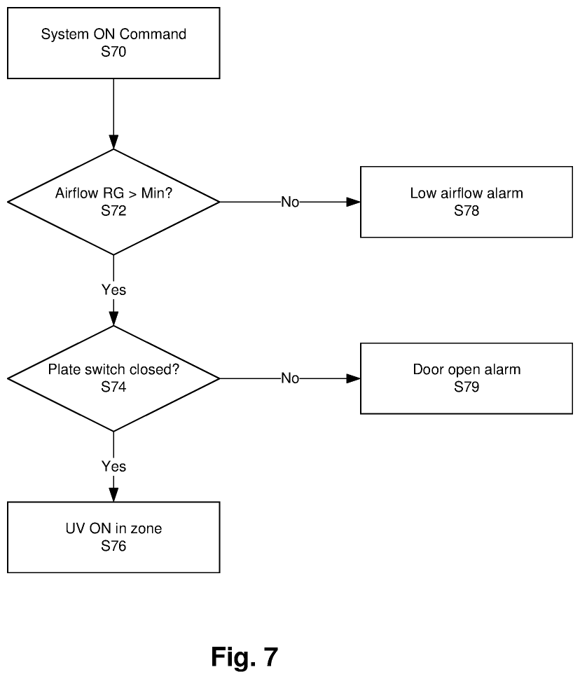

FIG. 7 illustrates an exemplary embodiment of a control flow for the disinfecting return grille 500 and 600. The system receives an ON command in S70. Next, the air flow rate inside the return grille is measured and compared to a predetermined threshold in S72. If the measured airflow is not higher than the predetermined threshold, a low airflow alarm is generated at S78. This may result in a message being output through a user interface.

If the measured airflow is sufficiently high, the process continues to S74, where a determination is made whether a plate switch 505 is closed. If the intake plate is determined to be opened, a door open alarm is output at S79. If the intake plate is determined to be closed, the UV light 120 in the return grille is powered on in S76. This process may continue on a schedule, or each of steps S72 and S74 can be continuously repeated. For example, when the HVAC system 530 shuts down, the air flow measurement in S72 will drop and the process will terminate with the low airflow alarm.

Referring to FIG. 8, a typical room 900 is illustrated with various embodiments of the preceding disclosure. A human occupant 990 is shown in the room 900 near a desk 992, and two dual-mode light fixtures 300 are installed along with a disinfecting return grille 500. An IR sensor 104 detects movement in the room and thus the presence or absence of a human occupant, in conjunction with magnet 106 and door sensor 105 which are installed on or near door 901. The controller 901 may detect motion in the room and the state (open or closed) of the door. If the door is closed and motion is detected, the room is deemed to be occupied, regardless of whether any subsequent motion is detected by IR sensor 104. The human occupant may sit motionless which could lead to the determination that the room is unoccupied, but that determination is not reached if the door remains closed. If the door is opened and closed, and immediately after the closing there is no motion detected in the room, the room is determined to be unoccupied. In this case, the combined system in FIG. 8 may begin the unoccupied mode to sanitize surfaces in the room by exposing the UV lights 120 to the room as described above. Of course, the unoccupied mode may be delayed based on a timer and only initiated if the time of day is within a prescribed range (e.g., outside of business hours) and the room is determined to be unoccupied.

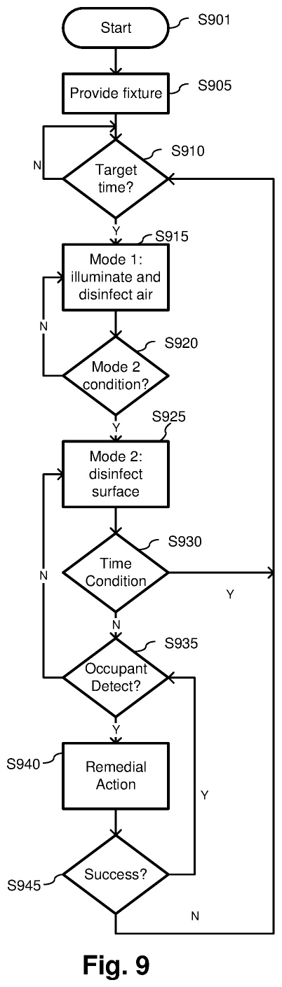

Referring to FIG. 9, an exemplary process for controlling a dual-mode fixture is shown. The process begins at S901, and can be run continuously. Processor 801 may be configured to execute the process of FIG. 9 as part of the basic configuration 801. The process applies too all embodiments of dual-mode light fixtures and ventilation grilles described in this disclosure. At S905, a fixture, such as dual-mode light fixture 100 is provided. The fixture is installed at its intended location, such as at a ceiling of a space (e.g., room). Initially, the fixture can be in a first mode that illuminates surrounding space and, in some embodiments, also sanitizes air that flows through the light fixture. For example, as shown at S910, the process can monitor whether a target time is reached. In embodiments, the target time can correspond to working hours (e.g., 8 am-4 pm), or other time period(s), to automatically supply power to the light fixture. This provides an ability to automatically manage power consumption and save energy.

Initially, at S915, the fixture is placed into the first mode, as noted above. In this mode, visible light is emitted from light source 130. At the same time, air may be drawn into the light fixture by fan 115 and exposed to disinfecting light radiation (e.g., UV-C) inside of the light fixture, as indicated by the arrows in FIG. 1A. The process continues through S920, where one or more conditions can be selected to trigger the change of the fixture into a second mode. In embodiments, the conditions include a time of day and a day of the week. These days and times can be based on the expected occupancy of the space where the light fixture is installed. For example, if the space is an office space where the working day ends at 4 pm, the condition can be the time passing 4 pm. In other embodiments, the condition can be provided from a computer system, such as scheduling software (e.g., Microsoft Outlook™), such as in the case of a conference room. For example, it is common to use scheduling software to reserve a shared resource such as a conference room. In embodiments, the reservation of a room (e.g., conference room) where the light fixture is installed is provided to processor 801, which may define the second mode condition in S920 to be time during which the conference room is scheduled to be unoccupied. Similarly, the reservation from the scheduling software may be provided to S910, to switch to the first mode and to turn on lights prior to human occupants arriving in the reserved room.

In further embodiments, the second mode condition in S920 may be based on the detection of the presence or absence of human occupants of the space in the vicinity of the light fixture and be independent of the time of day. For example, one or more sensors (motion sensor, acoustic sensors, pressure sensors) may be provided in and near the space where the light fixture is installed. The signals from these sensors are used to determine whether any human occupants are present, and if they are not present, and after some predetermined amount of time passes during which no human occupants are detected (e.g., 10 minutes, 5 minutes), the fixture transitions to the second mode at S925. The second mode allows disinfecting radiation (e.g., UV-C) to be emitted outside of the light fixture onto surfaces in the room where the fixture is installed, as described above and illustrated, for example, in FIGS. 2A, 2B, 4B.

While the fixture is in the second mode, it is desirable to ensure that no human occupants enter the space that is exposed to the disinfecting radiation. At S930, a time condition is checked to determine whether the second mode is finished. In embodiments, the time condition is an elapsed time. For example, the elapsed time may be set as a duration that is sufficient to reduce surface pathogens on surfaces exposed to the disinfecting light to an acceptable level. In other embodiments, the time condition is an absolute time of day, such as the time when human occupants are expected to return to the occupied space. In embodiments, the time is provided by scheduling software that is used to reserve the space for use by occupants.

If the time condition is met, the process continues at S910, as described above. If the time condition at S930 is not met, the process continues to S935, where a determination is made whether human occupants have entered the space being disinfected. It will be understood that the loop from S925, S930, and S935 runs continuously while the fixture is in the second mode, as a safety precaution. The detection of occupants at S935 can be through motion sensors (passive infra-red sensor, light beam sensors that detect the interruption of a light beam, door sensors such as magnet/reed-switch, pressure sensors on the floor, and others). If a determination is made at S935 that a human occupant is present, the process continues to S940.

At S940, remedial action can take place, that includes turning off the source of the disinfecting radiation. The light fixture itself may remain in the configuration of the second mode, so that the second mode of operation can be quickly resumed. The remedial action may include issuing audible warnings through speakers mounted in the light fixture (not illustrated) or installed in the room. The audible warning may indicate that a disinfecting operation is taking place, and request the occupant(s) to exit the space. A visible warning may also be generated on display terminal(s) in the room, or projected onto the ground from the light fixture using an optional built-in projector 8501. The projector 8501 may be installed on an outer surface of a fixture 500 as shown in FIG. 8, and have a light output port that projects a message and/or an image onto a surface. In embodiments, a projector 8501 may be installed inside of the light fixture such that it is concealed until the light fixture is transformed into the second mode.

After the remedial action S940, the process continues to S945, where a determination is made whether the remedial action was successful—namely effective at curing the condition that (e.g., presence of occupant) that caused the interruption. This determination can be the same or similar as S935. If it is determined to be successful, the process returns to S935 for another confirmation that no occupant is present in the space being disinfected, as described above.

If it is determined that the remedial action was not successful at S945, the process continues to S910 where the time target is checked as described above, and the light fixture may be transformed into the first mode to provide illumination and/or disinfection of the air drawn through the light fixture.

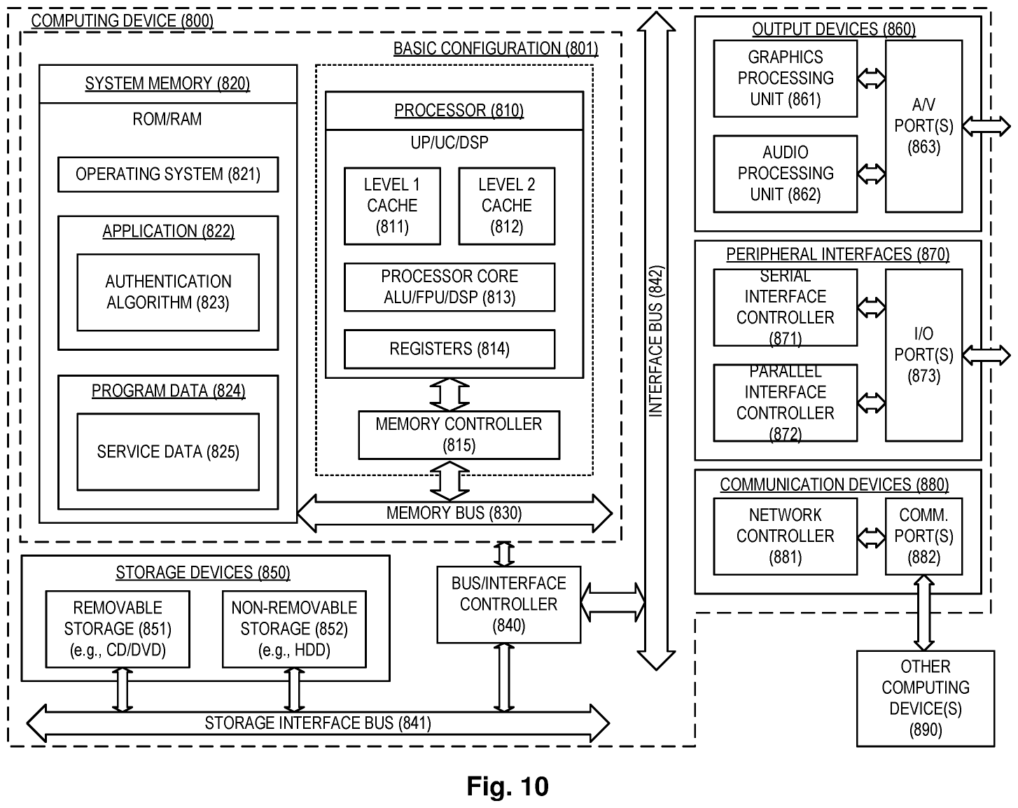

FIG. 10 illustrates an exemplary embodiment of the various controllers described above, embodied as a computing device 800. FIG. 10 is a block diagram illustrating an example computing device 800 that is arranged for controlling a dual-mode light fixtures, disinfecting return grilles, and/or HVAC systems in accordance with the present disclosure. In a very basic configuration 801, computing device 800 typically includes one or more processors 810 and system memory 820. A memory bus 830 can be used for communicating between the processor 810 and the system memory 820.

Depending on the desired configuration, processor 810 can be of any type including but not limited to a microprocessor (μP), a microcontroller (μC), a digital signal processor (DSP), or any combination thereof. Processor 810 can include one more levels of caching, such as a level one cache 811 and a level two cache 812, a processor core 813, and registers 814. The processor core 813 can include an arithmetic logic unit (ALU), a floating point unit (FPU), a digital signal processing core (DSP Core), or any combination thereof. A memory controller 815 can also be used with the processor 810, or in some implementations the memory controller 815 can be an internal part of the processor 810.

Depending on the desired configuration, the system memory 820 can be of any type including but not limited to volatile memory (such as RAM), non-volatile memory (such as ROM, flash memory, etc.) or any combination thereof. System memory 820 typically includes an operating system 821, one or more applications 822, and program data 824. Application 822 includes a multipath processing algorithm 823 that is arranged to control the light fixtures and the overall system according the disclosed embodiments. Program Data 824 includes data 825 that is useful for controlling a dual-mode light fixtures, disinfecting return grilles, and/or HVAC systems, as will be further described below. In some embodiments, application 822 can be arranged to operate with program data 824 on an operating system 821. This described basic configuration is illustrated in FIG. 10 by those components within dashed line 801.

Computing device 800 can have additional features or functionality, and additional interfaces to facilitate communications between the basic configuration 801 and any required devices and interfaces. For example, a bus/interface controller 840 can be used to facilitate communications between the basic configuration 801 and one or more data storage devices 850 via a storage interface bus 841. The data storage devices 850 can be removable storage devices 851, non-removable storage devices 852, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media can include volatile and nonvolatile, removable and non-removable media implemented in any method or technology for storage of information, such as computer readable instructions, data structures, program modules, or other data.

System memory 820, removable storage 851 and non-removable storage 852 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 800. Any such computer storage media can be part of device 800.

Computing device 800 can also include an interface bus 842 for facilitating communication from various interface devices (e.g., output interfaces, peripheral interfaces, and communication interfaces) to the basic configuration 801 via the bus/interface controller 840. Example output devices 860 include a graphics processing unit 861 and an audio processing unit 862, which can be configured to communicate to various external devices such as a display or speakers via one or more A/V ports 863. Example peripheral interfaces 870 include a serial interface controller 871 or a parallel interface controller 872, which can be configured to communicate with external devices such as input devices (e.g., keyboard, mouse, pen, voice input device, touch input device, etc.) or other peripheral devices (e.g., sensors 103.) via one or more I/O ports 873. An example communication device 880 includes a network controller 881, which can be arranged to facilitate communications with one or more other computing devices 890 over a network communication via one or more communication ports 882. The communication connection is one example of a communication media. Communication media may typically be embodied by computer readable instructions, data structures, program modules, or other data in a modulated data signal, such as a carrier wave or other transport mechanism, and includes any information delivery media. A modulated data signal can be a signal that has one or more of its characteristics set or changed in such a manner as to encode information in the signal. By way of example, and not limitation, communication media can include wired media such as a wired network or direct-wired connection, and wireless media such as acoustic, radio frequency (RF), infrared (IR) and other wireless media. The term computer readable media as used herein can include both storage media and communication media.

Computing device 800 can be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 800 can also be implemented as a personal computer including both laptop computer and non-laptop computer configurations.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

According to first embodiments, the disclosed subject matter includes a ventilation system return register that is mountable in a room from with air will flow through the return register. The return register includes a housing that defines an internal volume through which air can flow, a transition collar configured to attach to the ventilation system duct, an electrical junction box configured to receive an electrical connection, a disinfecting light source electrically connected to the electrical junction box and configured to emit a radiation in a range the destroys microbial contaminants, a reflective surface facing the disinfecting light and defining a disinfecting pathway through which air passes when the ventilation system is operating, and an intake plate covering the disinfecting light source and preventing light emitted from the disinfecting light source to shine into the room when the intake plate is in a closed position. A return air flow path is defined from the intake plate through the disinfecting pathway to the internal volume of the housing and through the transition collar into the ventilation system duct.

In other examples of the first embodiments, the return register includes a filter bracket configured to hold at least one filter between the intake plate and the transition ring. In still other examples, the return register includes a filter held by the filter bracket, wherein at least one surface of the filter is exposed to radiation emitted by the disinfecting light. In still other examples, the return register includes a pivot bracket rotatably mounted to the housing such that the pivot bracket can pivot about an axle, wherein the intake plate is attached to the pivot bracket at one end of the intake plate.

In still other examples, the return register includes at least one latch on the housing configured to old the intake plate in the closed position when the latch is closed, and to permit the intake plate to rotate to an opened position when the latch is opened.

In still other examples, the intake plate is configured to move between the closed position and an opened position, when the intake plate is in the closed position, radiation from the disinfecting light does not emit outside of the return register into space from which the return register is configured to receive return air, and when the intake plate is in the opened position, radiation from the disinfecting light radiates into the space from which the return register is configured to receive return air.

In still other examples, the return register includes a powered mechanism that that engages the pivot bracket to rotate the pivot bracket. In still other examples, the return register includes the powered mechanism is electrically connected to the electrical junction box to receive electricity and control signals, the powered mechanism is configured to move the intake plate between an opened position and the closed position in response to the control signals.

In still other examples, the return register includes a controller electrically connected through the electrical junction box to the powered mechanism and configured to output control signals that command the powered mechanism to move between the opened and closed position, one or more sensors configured to detect conditions in the space from which the return register is configured to receive return air and to output one or more sensor signals, a user interface configured to receive input commands from a user, wherein the controller is configured to receive at least the one or more sensor signals and the input commands and to command the powered mechanism based on the one or more sensor signals and the input commands.

In still other examples, the controller is further configured to receive a first sensor signal that indicates an airflow quantity through the return register, the controller is further configured to receive a sensor signal that indicates whether a door into the room is closed, the controller is further configured to determine whether the airflow quantity exceeds a first threshold, and the controller is further configured to turn on the disinfecting light source when it determines that the airflow quantity exceeds the first threshold and the door into the room is closed, and to turn off the disinfecting light source off otherwise.

In still other examples, the sensors include a proximity sensor, an infrared sensor, a magnetic sensor, a reed switch, an acoustic sensor, a temperature sensor, a pressure sensor, an airflow velocity sensor, an airflow volume sensor, a capacitance sensor, and an optical sensor.

In still other examples, the light source includes an ultra-violet (UV) light that is configured to emit light in the frequency range of 240-280 nm.

In still other examples, the return register includes a fan configured to generate a flow of air through the disinfecting pathway and into the transition collar and a drive mechanism that powers the fan. In still other examples, the controller is configured to output a control signal to the drive mechanism and thereby control a speed of the fan, and the controller is configured to control the speed of the fan in response to a pressure signal indicating a pressure in the ventilation system duct.

In still other examples, the return register includes the transition collar is fluidly connected to the ventilation system duct, the ventilation system duct is configured to convey return air from the return register to an air handler of a ventilation system that includes a ventilation system controller, the ventilation system controller is operatively connected to the controller of the return register and is configured to receive and transmit signals from and to the controller of the return register.

According to second embodiments, a ventilation system for an enclosed space includes an air treatment system that receives air at an intake plenum, treats the air, and outputs treated air at an outlet plenum. It further includes at least one supply duct connected to the outlet plenum and conveying the treated air toward the enclosed space, and at least one return duct connected to the intake plenum and conveying return air from the enclosed space to the intake plenum. It further includes one or more sensors configured to detect conditions in at least the enclosed space, the supply duct, or the return duct and a controller configured to receive a signal from the one or more sensors. It further includes a return register mounted in the enclosed space. The return register includes a housing that defines an internal volume through which return air can flow, a transition collar attached to the return duct, an electrical junction box configured to receive an electrical connection, a disinfecting light source electrically connected to the electrical junction box and configured to emit a radiation in a range that destroys microbial contaminants, a reflective surface facing the disinfecting light and defining a disinfecting pathway through which air passes when the ventilation system is operating, and an intake plate covering the disinfecting light source and preventing light emitted from the disinfecting light source to shine into the room when the intake plate is in a closed position. Further, a return air flow path is defined from the intake plate through the disinfecting pathway to the internal volume of the housing and through the transition collar into the return duct.

According to third embodiments, a light fixture is mountable on an internal surface of an enclosed space and includes a housing that includes a disinfecting plenum and an intake plenum, the intake plenum having an air inlet and a fan and fluidly connected to the disinfecting plenum. The fan is configured to draw from the enclosed space through the air inlet into the intake plenum and through the disinfecting plenum and the disinfecting plenum is defined by a portion of the housing at one side of the disinfecting plenum and by at least one rotating plate on an opposite side of the disinfecting plenum, the disinfecting plenum extending from the intake plenum to an air outlet. The at least one rotating plate is rotatably mounted within the housing and configured to rotate about a rotation axis, the at least one rotating plate having a first side and a second side opposed to the first side, a disinfecting light source mounted on the first side of the rotating plate, and a visible light source mounted on the second side of the rotating plate.

According to fourth embodiments, an illumination and ventilation system for an enclosed space includes a light fixture mounted on an internal surface of an enclosed space, the light fixture including a housing that includes a disinfecting plenum and an intake plenum, the intake plenum having an air inlet and a fan and fluidly connected to the disinfecting plenum. The fan is configured to draw from the enclosed space through the air inlet into the intake plenum and through the disinfecting plenum. The disinfecting plenum is defined by a portion of the housing at one side of the disinfecting plenum and by at least one rotating plate on an opposite side of the disinfecting plenum, the disinfecting plenum extending from the intake plenum to an air outlet. The at least one rotating plate is rotatably mounted within the housing and configured to rotate about a rotation axis, the at least one rotating plate having a first side and a second side opposed to the first side, and a disinfecting light source is mounted on the first side of the rotating plate, while a visible light source mounted on the second side of the rotating plate. A controller configured to control the fan, the disinfecting light source, and the visible light source. One or more sensors are configured to sense conditions in the enclosed space and to output sensor signals to the controller, wherein the internal surface is a ceiling of a room, a wall of a room, or a corner where the ceiling and the wall meet, and the controller is configured to control rotation of the rotating plate between an occupied mode and an unoccupied mode. In the occupied mode the disinfecting light source is enclosed within the disinfecting plenum and radiation from the disinfecting light source does not reach the enclosed space while the visible light source is positioned to emit visible light into the enclosed space. In the unoccupied mode the rotating plate is rotated until the disinfecting light source is exposed to the enclosed space and can emit radiation directly into the enclosed space in which the light fixture is mounted while the visible light source is positioned in the disinfecting plenum.

In examples of the fourth embodiments, the system further includes a ventilation system that has an air treatment system that receives air at an intake plenum, treats the air, and outputs treated air at an outlet plenum. At least one supply duct is connected to the outlet plenum and conveying the treated air toward the enclosed space and at least one return duct is connected to the intake plenum and conveys return air from the enclosed space to the intake plenum. A return register is mounted in the enclosed space, the return register including a housing that defines an internal volume through which return air can flow, a transition collar attached to the return duct, an electrical junction box configured to receive an electrical connection, a disinfecting light source electrically connected to the electrical junction box and configured to emit a radiation in a range that destroys microbial contaminants. A reflective surface faces the disinfecting light and defines a disinfecting pathway through which air passes when the ventilation system is operating and an intake plate covers the disinfecting light source and prevents light emitted from the disinfecting light source to shine into the room when the intake plate is in a closed position. A return air flow path is defined from the intake plate through the disinfecting pathway to the internal volume of the housing and through the transition collar into the return duct.

According to fifth embodiments, a method includes providing a dual-mode fixture that is configured to generate a first radiation with germicidal properties and a second radiation with illumination properties. The method also includes, at a first time, generating the first radiation with the germicidal properties in the dual-mode fixture and flowing air through the dual-mode fixture and exposing the air to the first radiation with the germicidal properties during the flowing, while substantially preventing the first radiation to irradiate surfaces outside of the dual-mode fixture. The method also includes at the first time, generating the second radiation with the illumination properties and irradiating surfaces outside of the dual-mode fixture with the second radiation.

In examples of the fifth embodiments, the method further includes at a second time that is different from the first time, changing a physical configuration of the dual-mode fixture to permit the first radiation with the germicidal properties to radiate outside of the dual-mode fixture and to permit the first radiation to irradiate surfaces outside of the dual-mode fixture.

In other examples of the fifth embodiments, the method further includes at the second time, discontinuing the generation of the second radiation with the illumination properties.

In yet other examples of the fifth embodiments, the first radiation has a wavelength in the ultraviolet frequency range, and the second radiation includes wavelengths in the visible light spectrum.

In yet other examples of the fifth embodiments, the first radiation has wavelengths in the range of 240-280 nm.

In yet other examples of the fifth embodiments, the method includes verifying absence of human occupants in a vicinity of the dual-mode fixture before the second time.

In yet other examples of the fifth embodiments, the method includes at a third time, distinct from the second time, detecting presence of a human occupant in a vicinity of the dual-mode fixture and discontinuing the generating of the first radiation in response to the detecting of the presence.

In yet other examples of the fifth embodiments, the method includes at a fourth time, distinct from the third time, verifying absence of human occupants in a vicinity of the dual-mode fixture and recommencing the generating of the first radiation in response to the verifying the absence.

In yet other examples of the fifth embodiments, the method includes at a fifth time, distinct from the fourth time, changing the physical configuration of the dual-mode fixture to substantially prevent the first radiation with the germicidal properties to radiate outside of the dual-mode fixture and recommencing the generation of the second radiation with the illumination properties.

In yet other examples of the fifth embodiments, the fifth time is an absolute time of time, and the method includes storing the fifth time in a controller memory.

It should be apparent that all of the embodiments of the lights, return grilles, and HVAC systems can be combined to produce further embodiments. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present disclosure.

1. A ventilation system return register that is mountable in a room from with air will flow through the return register, comprising:
a housing that defines an internal volume through which air can flow;
a transition collar configured to attach to a duct of the ventilation system;
an electrical junction box configured to receive an electrical connection;
a disinfecting light source electrically connected to the electrical junction box and configured to emit a radiation in a range that destroys microbial contaminants;
a reflective surface facing the disinfecting light and defining a disinfecting pathway through which air passes when the ventilation system is operating; and
an intake plate covering the disinfecting light source and preventing light emitted from the disinfecting light source to shine into the room when the intake plate is in a closed position, wherein
a return air flow path is defined from the intake plate through the disinfecting pathway to the internal volume of the housing and through the transition collar into the ventilation system duct.

2. The ventilation system return register according to claim 1, further comprising:
a filter bracket configured to hold at least one filter between the intake plate and the transition ring.

3. The ventilation system return register according to any of claims 1-2, further comprising:
a filter held by the filter bracket, wherein
at least one surface of the filter is exposed to radiation emitted by the disinfecting light.

4. The ventilation system return register according to claim 1, further comprising:
a pivot bracket rotatably mounted to the housing such that the pivot bracket can pivot about an axle, wherein
the intake plate is attached to the pivot bracket at one end of the intake plate.

5. The ventilation system return register according to claim 1, further comprising:
at least one latch on the housing configured to hold the intake plate in the closed position when the latch is closed, and to permit the intake plate to rotate to an opened position when the latch is opened.

6. The ventilation system return register according to claim 1, wherein
the intake plate is configured to move between the closed position and an opened position,
when the intake plate is in the closed position, the radiation from the disinfecting light does not emit outside of the return register into space from which the return register is configured to receive return air, and
when the intake plate is in the opened position, the radiation from the disinfecting light radiates into the space from which the return register is configured to receive the return air.

7. The ventilation system return register according to claim 4, further comprising:
a powered mechanism that that engages the pivot bracket to rotate the pivot bracket.

8. The ventilation system return register according to claim 7, wherein
the powered mechanism is electrically connected to the electrical junction box to receive electricity and control signals,
the powered mechanism is configured to move the intake plate between an opened position and the closed position in response to the control signals.

9. The ventilation system return register according to claim 8, further comprising:
a controller electrically connected through the electrical junction box to the powered mechanism and configured to output control signals that command the powered mechanism to move between the opened and the closed position;
one or more sensors configured to detect conditions in a space from which the return register is configured to receive return air and to output one or more sensor signals;
a user interface configured to receive input commands from a user, wherein
the controller is configured to receive at least the one or more sensor signals and the input commands and to command the powered mechanism based on the one or more sensor signals and the input commands.

10. The ventilation system return register according to claim 9, wherein
the controller is further configured to receive a first sensor signal that indicates an airflow quantity through the return register,
the controller is further configured to receive a sensor signal that indicates whether a door into the room is closed,
the controller is further configured to determine whether the airflow quantity exceeds a first threshold, and
the controller is further configured to turn on the disinfecting light source when it determines that the airflow quantity exceeds the first threshold and the door into the room is closed, and to turn off the disinfecting light source off otherwise.

11. The ventilation system return register according to claim 9, wherein the sensors include one or more of:
a proximity sensor, an infrared sensor, a magnetic sensor, a reed switch, an acoustic sensor, a temperature sensor, a pressure sensor, an airflow velocity sensor, an airflow volume sensor, a capacitance sensor, and an optical sensor.

12. The ventilation system return register according to claim 10, wherein
the disinfecting light source includes an ultra-violet (UV) light that is configured to emit light in a frequency range of 240-280 nm.

13. The ventilation system return register according to claim 12, further comprising:
a fan configured to generate a flow of air through the disinfecting pathway and into the transition collar; and
a drive mechanism that powers the fan.

14. The ventilation system return register according to claim 13, wherein
the controller is configured to output a control signal to the drive mechanism and thereby control a speed of the fan, and
the controller is configured to control the speed of the fan in response to a pressure signal indicating a pressure in the ventilation system duct.

15. The ventilation system return register according to claim 1, wherein
the transition collar is fluidly connected to the ventilation system duct,
the ventilation system duct is configured to convey return air from the return register to an air handler of a ventilation system that includes a ventilation system controller,
the ventilation system controller is operatively connected to the controller of the return register and is configured to receive and transmit signals from and to the controller of the return register.

16. A ventilation system for an enclosed space, the ventilation system comprising:
an air treatment system that receives air at an intake plenum, treats the air, and outputs treated air at an outlet plenum;
at least one supply duct connected to the outlet plenum and conveying the treated air toward the enclosed space;
at least one return duct connected to the intake plenum and conveying return air from the enclosed space to the intake plenum;
one or more sensors configured to detect conditions in at least the enclosed space, the supply duct, or the return duct;
a controller configured to receive a signal from the one or more sensors;
a return register mounted in the enclosed space, the return register including
a housing that defines an internal volume through which return air can flow;
a transition collar attached to the return duct;
an electrical junction box configured to receive an electrical connection;
a disinfecting light source electrically connected to the electrical junction box and configured to emit a radiation in a range that destroys microbial contaminants;
a reflective surface facing the disinfecting light and defining a disinfecting pathway through which air passes when the ventilation system is operating; and
an intake plate covering the disinfecting light source and preventing light emitted from the disinfecting light source to shine into the enclosed space when the intake plate is in a closed position, wherein
a return air flow path is defined from the intake plate through the disinfecting pathway to the internal volume of the housing and through the transition collar into the return duct.

17. The ventilation system according to claim 16, wherein
the controller is configured to turn on and off the disinfecting light source when it determines that an airflow through the return duct is above a predetermined threshold and a door into the enclosed space is closed.

18. The ventilation system according to claim 16, wherein
the controller is configured to turn on and off a fan in the return register.

19. A light fixture mountable on an internal surface of an enclosed space, the light fixture comprising:
a housing that includes a disinfecting plenum and an intake plenum;
the intake plenum having an air inlet and a fan and fluidly connected to the disinfecting plenum;
the fan configured to draw from the enclosed space through the air inlet into the intake plenum and through the disinfecting plenum;
the disinfecting plenum being defined by a portion of the housing at one side of the disinfecting plenum and by at least one rotating plate on an opposite side of the disinfecting plenum, the disinfecting plenum extending from the intake plenum to an air outlet;
the at least one rotating plate rotatably mounted within the housing and configured to rotate about a rotation axis, the at least one rotating plate having a first side and a second side opposed to the first side;
a disinfecting light source mounted on the first side of the rotating plate; and
a visible light source mounted on the second side of the rotating plate.

20. The light fixture according to claim 19, further comprising:
a light tube holder that extends through the rotating plate and holds the disinfecting light source on the first side and holds the visible light source on the second side.

21-46. (canceled)
